(19) United States
(12) Patent Application Publication
(10) Pub. No.: US 2022/0160235 A1
(43) Pub. Date: May 26, 2022

(54) Component Concentration Measuring Device

(71) Applicant: Nippon Telegraph and Telephone Corporation, Tokyo (JP)

(72) Inventors: Yujiro Tanaka, Tokyo (JP); Masahito Nakamura, Tokyo (JP); Daichi Matsunaga, Tokyo (JP); Michiko Seyama, Tokyo (JP)

(21) Appl. No.: 17/417,531

(22) PCT Filed: Dec. 10, 2019
(86) PCT No.: PCT/JP2019/048253

§ 371 Date: Jan. 12, 2022

(30) Foreign Application Priority Data
Dec. 25, 2018 (JP) 2018-240790

(51) Int. Cl. A61B 5/00; A61B 5/145; G01N 29/24; G01N 29/32

(52) U.S. Cl. CPC A61B 5/0095; A61B 5/7289; A61B 5/024; G01N 29/2418; G01N 29/32; A61B 5/14532

(57) ABSTRACT

A measuring unit (103) chronologically measures a heart rate or pulse rate of a measurement subject. A computing unit (104) obtains a statistic of measuring results measured by the measuring unit (103). The computing unit (104) obtains a statistic of a plurality of measuring results measured during a set period of time by the measuring unit (103) and stored in a storage unit. A correction unit (105) corrects a photoacoustic signal detected by a detection unit (102) based on the statistic obtained by the computing unit (104). The correction unit (105) corrects the photoacoustic signal that is detected by the detection unit (102) immediately after the latest value of the heart rate or pulse rate used in the calculation of the statistic by the computing unit (104) is measured. The correction unit (105) performs the correction by adding a value obtained by multiplying the statistic obtained by the computing unit (104) by a pre-set correction coefficient, to the photoacoustic signal detected by the detection unit (102).

### CROSS-REFERENCE TO RELATED APPLICATIONS

This application is a national phase entry of PCT Application No. PCT/JP2019/048253, filed on Dec. 10, 2019, which claims priority to Japanese Application No. 2018-240790, filed on Dec. 25, 2018, which applications are hereby incorporated herein by reference.

### TECHNICAL FIELD

The present invention relates to a component concentration measuring device, and more specifically relates to a component concentration measuring device for non-invasively measuring the concentration of a component such as glucose in blood.

### BACKGROUND

Knowing (measuring) the blood glucose level is very important when determining an insulin dosage for a person with diabetes, preventing diabetes, and so on. The blood glucose level is the concentration of glucose in blood, and photoacoustics is a well-known method for measuring the concentration of this type of component (see PTL 1).

When a living body is irradiated with a certain amount of light (electromagnetic waves), the emitted light is absorbed by molecules of the living body. For this reason, measurement target molecules in the portion irradiated with light are locally heated and expand, thus emitting acoustic waves. The pressure of such acoustic waves is dependent on the quantity of molecules that absorb the light. Photoacoustics is a method of measuring a molecular quantity in a living body by measuring such acoustic waves (a photoacoustic signal). Acoustic waves are pressure waves that propagate in a living body and have a characteristic of undergoing less diffusion than electromagnetic waves, and therefore photoacoustics can be said to be suited to the measurement of a blood component in a living body.

Photoacoustic measurement makes it possible to continuously monitor the glucose concentration in blood. Furthermore, photoacoustic measurement does not require a blood sample, and does not cause the measurement subject discomfort.

### CITATION LIST

### Patent Literature

PTL 1 Japanese Patent Application Publication No. 2010-104858

### Non Patent Literature

NPL 1 Kei Kuwabara et al, “Blood Flow Observed with Smartphone—Ultracompact Wearable Blood Flow Sensor”, NTT Gizyutu Journal, pp. 21-24, November 2014

### SUMMARY

### Technical Problem

Meanwhile, in a human body site serving as a subject of this type of measurement, the diameters of blood vessels are not consistently constant but are varying. For example, when the blood flow rate of a person is measured, in addition to the heart rate, periodic fluctuations at intervals of about 10 seconds are perceived even when the person is at rest. Those fluctuations at intervals of about 10 seconds shows an influence of constriction motion of blood vessels, which is called vasomotion. If the diameter of a blood vessel is changed due to such constriction motion of the blood vessel, the ratio of plasma in blood and interstitial fluid at this position will change, leading to measurement error.

Embodiments of the present invention were achieved in order to solve the foregoing problems, and an object of embodiments of the present invention is to suppress measurement error caused by a change in the state of a blood vessel when measuring the concentration of a component such as glucose in a human body using photoacoustics.

### Means for Solving the Problem

A component concentration measuring device according to embodiments of the present invention includes: a light emitting unit configured to irradiate a measurement site of a measurement subject with a light beam having a wavelength that is absorbed by a measurement target substance; a detection unit configured to chronologically detect a photoacoustic signal generated in the measurement site irradiated with the light beam; a measuring unit configured to chronologically measure a heart rate or pulse rate of the measurement subject; a computing unit configured to obtain a statistic of a plurality of values of the heart rate or pulse rate chronologically measured by the measuring unit; and a correction unit configured to correct the photoacoustic signal detected by the detection unit, based on the statistic obtained by the computing unit.

In a configuration example of the component concentration measuring device, the correction unit corrects the photoacoustic signal that is detected by the detection unit immediately after the latest value of the heart rate or pulse rate used in the calculation of the statistic by the computing unit is measured.

In a configuration example of the component concentration measuring device, the correction unit performs the correction by adding a value obtained by multiplying the statistic obtained by the computing unit by a pre-set correction coefficient to the photoacoustic signal detected by the detection unit.

In a configuration example of the component concentration measuring device, the computing unit obtains, as the statistic, any of a dispersion, a standard deviation, an average, and a temporal differential value of the plurality of values of the heart rate or pulse rate chronologically measured by the measuring unit.

In a configuration example of the component concentration measuring device, a concentration calculation unit configured to obtain a concentration of the substance based on the photoacoustic signal corrected by the correction unit is further provided.

In a configuration example of the component concentration measuring device, the substance is glucose, the substance is glucose, and the light emitting unit emits the light beam having a wavelength that is absorbed by glucose.

### Effects of Embodiments of the Invention

As described above, according to embodiments of the present invention, since a detected photoacoustic signal is corrected based on a statistic of values of the heart rate or pulse rate of a measurement subject that were measured chronologically, an excellent effect can be achieved that when measuring the concentration of a component such as glucose in a human body using photoacoustics, it is possible to suppress error in the component concentration measurement caused by a change in the state of a blood vessel.

### BREIF DESCRIPTION OF THE DRAWINGS

FIG. 1 is a configuration diagram illustrating a configuration of a component concentration measuring device according to an embodiment of the present invention.

FIG. 2 is a configuration diagram illustrating in more detail the configuration of the component concentration measuring device according to the embodiment of the present invention.

FIG. 3 is a configuration diagram illustrating hardware configurations of a computing unit 104, a correction unit 105, and a concentration calculation unit 106 of the component concentration measuring device according to embodiments of the present invention.

### DETAILED DESCRIPTION OF ILLUSTRATIVE EMBODIMENTS

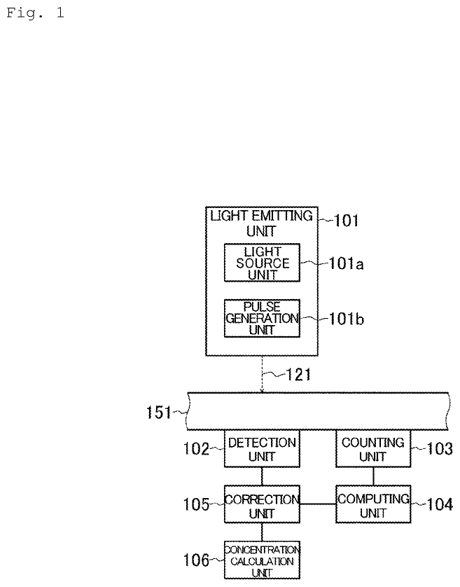

The following describes a component concentration measuring device according to an embodiment of the present invention, with reference to FIG. 1. The component concentration measuring device includes a light emitting unit 101, a detection unit 102, a measuring unit 103, a computing unit 104, a correction unit 105, and a concentration calculation unit 106.

The light emitting unit 101 generates a light beam 121 having a wavelength that is absorbed by a measurement target substance, and emits the generated light beam 121 from a first holding member 111 toward a measurement site 151. For example, in the case where the measurement target substance is glucose in blood, the light emitting unit 101 includes a light source unit 101a that generates the light beam 121 having a wavelength that is absorbed by glucose, and a pulse generation unit 101b that converts the light beam 121 generated by the light source into pulsed light that has a pre-set pulse width.

Note that glucose exhibits a property of absorbing light in wavelength bands near 1.6 μm and 2.1 μm (see PTL 1). If glucose is the measurement target substance, the light beam 121 emitted by the light emitting unit 101 is a light beam having a pulse width of 0.02 seconds or longer.

The detection unit 102 is housed in a second holding member 112. The detection unit 102 detects a photoacoustic signal generated in the measurement site 151 that was irradiated with the light beam 121. The detection unit 102 detects a photoacoustic signal every two minutes, for example. The detection unit 102 can be a unit that employs a piezoelectric effect or an electrostrictive effect (e.g., a crystal microphone, a ceramic microphone, or a ceramic ultrasonic sensor), a unit that employs electromagnetic induction (e.g., a dynamic microphone or a ribbon microphone), a unit that employs an electrostatic effect (e.g., a condenser microphone), or a unit that employs magnetostriction (e.g., a magnetostrictive vibrator). For example, in the case of employing a piezoelectric effect, the unit includes a crystal made of a frequency flat-type electrostrictive element (ZT) or PVDF (polyvinylidene fluoride). The detection unit 102 can also be constituted by a PZT that includes an FET (Field Effect Transistor) amplifier. The detection unit 102 may store, in a storage unit 105, the photoacoustic signal together with information regarding the time when the signal was measured.

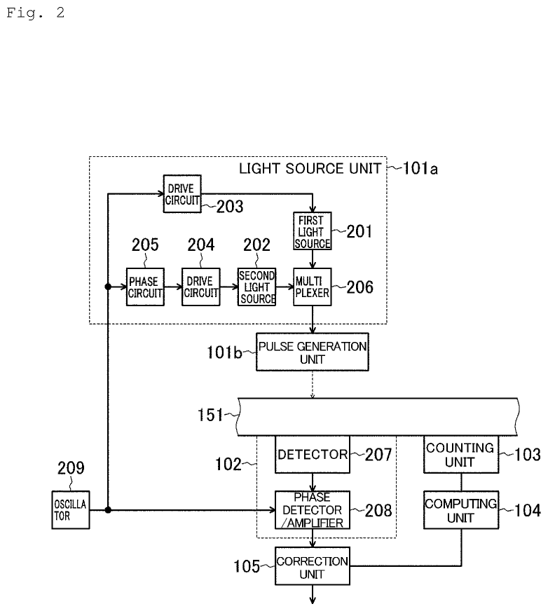

The following is a more detailed description of the light emitting unit 101 and the detection unit 102 with reference to FIG. 2. First, the light source unit 101a includes a first light source 201, a second light source 202, a drive circuit 203, a drive circuit 204, a phase circuit 205, and a multiplexer 206. Also, the detection unit 102 includes a detector 207, a phase detector/amplifier 208, and an oscillator 209.

The oscillator 209 is connected to the drive circuit 203, the phase circuit 205, and the phase detector/amplifier 208 via signal lines. The oscillator 209 transmits signals to the drive circuit 203, the phase circuit 205, and the phase detector/amplifier 208.

The drive circuit 203 receives the signal transmitted from the oscillator 209, and supplies a drive voltage to the first light source 201 so as to cause the first light source 201 to emit light whose intensity has been modulated in synchronization with the frequency of the signal. The first light source 201 is, for example, a semiconductor laser.

The phase circuit 205 receives the signal transmitted from the oscillator 209, changes the phase of the received signal by 180 degrees, and transmits the resultant signal to the drive circuit 204 via a signal line.

The drive circuit 204 receives the signal transmitted from the phase circuit 205, and supplies a drive voltage to the second light source 202 so as to cause the second light source 202 to emit light whose intensity has been modulated at the frequency of the above-described signal and in synchronization with the signal subjected to the phase change of 180 degrees by the phase circuit 205. The second light source 202 is, for example, a semiconductor laser.

The first light source 201 and the second light source 202 respectively output light beams that have mutually different wavelengths, and the light beams output by the light sources are each guided to the multiplexer 206 by an optical wave transmitting means. The wavelengths of the first light source 201 and the second light source 202 are set such that the wavelength of one of the light beams is a wavelength absorbed by glucose, and the wavelength of the other light beam is a wavelength absorbed by water. Also, the wavelengths of the light sources are set so that the degrees of absorption of the light beams are equal to each other.

The light beam output by the first light source 201 and the light beam output by the second light source 202 are multiplexed into a single light beam in the multiplexer 206, and the single light beam is then incident on the pulse generation unit 101b. The pulse generation unit 101b can be constituted by an optical chopper, for example. Upon receiving the light beam, the pulse generation unit 101b converts the incident light beam into pulsed light that has a predetermined pulse width, and emits the pulsed light toward the measurement site 151.

The detector 207 detects a photoacoustic signal generated at the measurement site 151, converts the detected photoacoustic signal into an electric signal, and transmits the electric signal to the phase detector/amplifier 208 via a signal line. The phase detector/amplifier 208 receives a synchronization signal necessary for synchronous detection from the oscillator 209, and receives the electric signal that is proportional to the photoacoustic signal and was transmitted from the detector 207, performs synchronous detection, amplification, and wave filtering, and outputs the resultant electric signal that is proportional to the photoacoustic signal. The electric signal (photoacoustic signal) thus processed is stored in the storage unit (not shown) together with information regarding the time when the electric signal was measured.

The intensity of the signal output from the phase detector/amplifier 208 is proportional to the amounts of light that were absorbed by components (glucose and water) in the measurement site 151 and respectively output by the first light source 201 and the second light source 202, and thus the intensity of the signal is proportional to the amounts of such components at the measurement site 151. The correction unit 105 corrects the measured value of the intensity of the signal (photoacoustic signal) output in this way, and the concentration calculation unit 106 calculates, based on the corrected photoacoustic signal, the component amount (concentration) of the measurement target substance (glucose) in blood within the measurement site 151.

As described above, two beams of light that have been subjected to intensity modulation based on signals having the same frequency are used in order to eliminate the influence of the non-uniformity of frequency characteristics when using a plurality of light beams, which is problematic when intensity modulation is performed based on signals having a plurality of frequencies.

On the other hand, nonlinear absorption coefficient dependence of a photoacoustic signal, which is problematic in measurement using photoacoustics, can be resolved by performing the measurement using light beams that have different wavelengths but have the same absorption coefficient as described above (see PTL 1).

Then, the measuring unit 103 chronologically measures the heart rate or pulse rate of the measurement subject. The measuring unit 103 measures, for example, the heart rate per minute every 10 seconds. The measuring unit 103 can be constituted by, for example, a laser rheometer (see NPL 1). The laser rheometer irradiates the skin with infrared light emitted from a laser light source, and detects scattered light using a light receiving element. Due to the doppler phenomenon of light, light that collides with red blood cells moving in a blood vessel and scatters is subjected to frequency shift that is proportional to the moving speed of the red blood cells. Therefore, by analyzing the frequency spectrum of a detected signal, it is possible to acquire information (pulse rate) regarding the flow of the blood. The measuring unit 103 can also be constituted by an electrocardiograph. The heart rate can be obtained based on the electrocardiographic potential measured by the electrocardiograph.

The computing unit 104 obtains a statistic of a plurality of values (measured values) of the heart rate or pulse rate chronologically measured by the measuring unit 103. The computing unit 104 obtains a statistic of a plurality of measured values that were measured during a set period of time by the measuring unit 103, and are stored in the storage unit. For example, the computing unit 104 obtains a dispersion of the plurality of measured values as the statistic. Also, for example, the computing unit 104 obtains a standard deviation of the plurality of measured values as the statistic. Also, for example, the computing unit 104 obtains an average of the plurality of measured values as the statistic. Also, for example, the computing unit 104 obtains a temporal differential value of the plurality of measured values as the statistic.

The period of time for measuring the plurality of measured values that are used to obtain a statistic is set to be longer than the cycle of the constriction motion of blood vessels. For example, the vasomotion occurs every 3 seconds, and thus the period of time for measuring a plurality of measured values that are used to obtain a statistic is set to 3 seconds or longer.

The correction unit 105 corrects the photoacoustic signal detected by the detection unit 102, based on the statistic obtained by the computing unit 104. The correction unit 105 corrects the photoacoustic signal that is detected by the detection unit 102 immediately after the latest value of the heart rate or pulse rate used in the calculation of the statistic by the computing unit 104 is measured. In the embodiment, the correction unit 105 performs the correction by adding a value obtained by multiplying the statistic obtained by the computing unit 104 by a pre-set correction coefficient, to the photoacoustic signal detected by the detection unit 102. The correction coefficient is a coefficient for correcting the time needed for blood glucose to reach sites by obtaining the blood flow level from the pulse rate.

The correction coefficient can be obtained in advance as will be described below. A statistic of a plurality of measured values counted by the measuring unit 103 in a predetermined period of time is obtained. Also, the concentration value of the measurement target substance (e.g., glucose) is measured by analyzing the blood actually collected in the above-described predetermined period of time. Then, the correction coefficient is set so that the concentration value that is calculated by the concentration calculation unit 106 based on a corrected value obtained by the correction unit 105 correcting a photoacoustic signal detected by the detection unit 102 immediately after the above-described predetermined period of time is equal to the concentration value measured through the analysis of the blood.

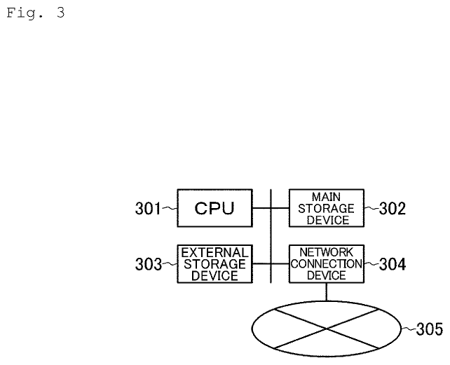

Note that the above-described computing unit 104, correction unit 105, and concentration calculation unit 106 are realized by a computer apparatus that includes, as shown in FIG. 3, a CPU (Central Processing Unit) 301, a main storage device 302, an external storage device 303, a network connection device 304 for connecting to a network 305, and the like. The above-described functions are realized by a program expanded on the main storage device 302 causing the CPU 301 to operate. Also, the functions may be distributed among a plurality of computer apparatuses.

As described above, according to embodiments of the present invention, since a detected photoacoustic signal is corrected based on a statistic of values of the heart rate or pulse rate of a measurement subject that were measured chronologically, it is possible to suppress error in component concentration measurement caused by a change in the state of a blood vessel.

Note that the present invention is not limited to the above-described embodiment, and it is apparent that various modifications and combinations can be implemented by a person skilled in the art to which the present invention pertains without departing from the technical idea of the present invention.

### REFERENCE SIGNS LIST

101 Light emitting unit

101a Light source unit

101b Pulse control unit

102 Detection unit

103 Measuring unit

104 Computing unit

105 Correction unit

106 Concentration calculation unit.

1.-6. (canceled)

7. A component concentration measuring device comprising:
a light emitting device configured to irradiate a measurement site of a measurement subject with a light beam having a wavelength that is absorbed by a measurement target substance;
a detector configured to chronologically detect a photoacoustic signal generated in the measurement site that is irradiated with the light beam;
a measuring circuit configured to chronologically measure a heart rate or a pulse rate of the measurement subject;
a processor configured to obtain a statistic of a plurality of values of the heart rate or the pulse rate chronologically measured by the measuring circuit; and
a correction circuit configured to correct the photoacoustic signal detected by the detector based on the statistic obtained by the processor.

8. The component concentration measuring device according to claim 7, wherein
the correction circuit is further configured to correct the photoacoustic signal that is detected by the detector immediately after a latest value of the heart rate or the pulse rate used in calculating the statistic by the processor is measured.

9. The component concentration measuring device according to claim 7, wherein:
the correction circuit is configured to correct the photoacoustic signal by adding a value obtained by multiplying the statistic obtained by the processor by a pre-set correction coefficient to the photoacoustic signal detected by the detector.

10. The component concentration measuring device according to claim 7, wherein:
the processor is configured to obtain, as the statistic, a dispersion, a standard deviation, an average, or a temporal differential value of the plurality of values of the heart rate or the pulse rate chronologically measured by the measuring circuit.

11. The component concentration measuring device according to claim 7, further comprising a concentration calculator configured to obtain a concentration of the substance based on the photoacoustic signal corrected by the correction circuit.

12. The component concentration measuring device according to claim 7, wherein the measurement target substance is glucose, and the light emitting device is configured to emit the light beam having a wavelength that is absorbed by glucose.

13. A method comprising:
irradiating, by a light emitting device, a measurement site of a measurement subject with a light beam having a wavelength that is absorbed by a measurement target substance;
chronologically detect a photoacoustic signal generated in the measurement site that is irradiated with the light beam;
chronologically measure a heart rate or a pulse rate of the measurement subject;
obtain, by a processor, a statistic of a plurality of values of the heart rate or the pulse rate that is chronologically measured; and
correcting the photoacoustic signal based on the statistic to obtain a corrected photoacoustic signal.

14. The method according to claim 13, wherein correcting the photoacoustic signal comprises correcting the photoacoustic signal immediately after a latest value of the heart rate or the pulse rate used in calculating the statistic is measured.

15. The method according to claim 13, wherein correcting the photoacoustic signal comprises adding a value obtained by multiplying the statistic by a pre-set correction coefficient to the photoacoustic signal.

16. The method according to claim 13, wherein obtaining the statistic comprises obtaining, as the statistic, a dispersion, a standard deviation, an average, or a temporal differential value of the plurality of values of the heart rate or the pulse rate that is chronologically measured.

17. The method according to claim 13, further comprising obtaining a concentration of the substance based on the corrected photoacoustic signal.

18. The method according to claim 13, wherein the measurement target substance is glucose, and the light emitting device is configured to emit the light beam having a wavelength that is absorbed by glucose.
